(19) United States
(12) Patent Application Publication
(10) Pub. No.: US 2024/0306908 A1
(43) Pub. Date: Sep. 19, 2024

(54) A METHOD TO DETECT RETINAL AMYLOIDOSIS AND TAUOPATHY USING SNAP HYPERSPECTRAL IMAGING AND/OR SNAP HYPERSPECTRAL OPTICAL COHERENCE TOMOGRAPHY

(71) Applicants: CEDARS-SINAI MEDICAL CENTER, Los Angeles, CA (US); THE REGENTS OF THE UNIVERSITY OF CALIFORNIA, Oakland, CA (US)

(72) Inventors: Maya Koronyo, Los Angeles, CA (US); Yosef Koronyo, Los Angeles, CA (US); Keith L. Black, Los Angeles, CA (US); Nazanin Mirzaei, West Hills, CA (US); Liang Gao, Los Angeles, CA (US)

(21) Appl. No.: 18/577,161

(22) PCT Filed: Jul. 8, 2022
(86) PCT No.: PCT/US2022/073558

§ 371 Date: Jan. 5, 2024

Related U.S. Application Data
(60) Provisional application No. 63/220,239, filed on Jul. 9, 2021.

(51) Int. Cl. A61B 3/10; A61B 3/12; G16H 30/20; G16H 50/20

(52) U.S. Cl. CPC A61B 3/102; A61B 3/12; G16H 30/20; G16H 50/20

(57) ABSTRACT

The present invention describes systems and method for the detection, diagnosis and monitoring of cognitive impairment and Alzheimer's disease. In one example, an integrated system comprising a full-field Fourier domain optical coherence tomography and an image mapping spectrometry is used for generating spectrally resolved volumetric images at wavelengths that show difference in spectral signatures of normal cells compared to spectral signatures of amyloid beta and pTau deposits, and also image inner retinal layers where amyloid beta and pTau deposits may aggregate.

### CROSS-REFERENCE TO RELATED APPLICATIONS

This application claims priority to and the benefit of U.S. Provisional Patent Application No. 63/220,239, filed Jul. 9, 2021, which is hereby incorporated by reference herein in its entirety.

### STATEMENT REGARDING FEDERALLY SPONSORED RESEARCH OR DEVELOPMENT

This invention was made with Government support under Grant Nos. AG056478, AG055865, and EY029397 awarded by the National Institutes of Health. The Government has certain rights in the invention.

### FIELD OF INVENTION

This invention relates to the detection of and monitoring of cognitive impairment; for example, related with Alzheimer's disease.

### BACKGROUND AND SUMMARY

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The retina is a developmental outgrowth of the brain and the only part of the central nervous system that can be noninvasively imaged at a high spatial resolution. Pathological hallmarks of Alzheimer's Disease (AD)—the abnormal deposition of amyloid β-protein (Aβ) and hyperphosphorylated pTau protein aggregates have been identified in the retinas of AD patients, including in early-stage patients with mild cognitive impairment (MCI). Aβ pathology, especially accumulation of the amyloidogenic 42-residue long alloform (Aβ42), is considered the earliest, most specific sign of AD, and together with tauopathy, defines AD diagnosis. Further, the Aβ40 alloform is a main constituent of cerebral amyloid angiopathy (CAA), a typical feature of AD, which was also identified in retinal blood vessels of MCI and AD patients. Hence, the specific detection of retinal Aβ40 and Aβ42, vascular Aβ, and pTau deposits may allow for large-scale screening and monitoring of at-risk populations and potentially assessing therapeutic responses.

Visualization of retinal Aβ and pTau deposits is non-trivial because conventional fundus photography provides little contrast. To increase visibility, state-of-the-art methods use exogenous fluorophores. However, administrating contrast agents in humans complicates the imaging procedure, hindering its scalability for population screening. To date, only curcumin, a natural fluorochrome, has been tested and used in clinical trials to label retinal Aβ; fluorophores used to visualize retinal pTau in vivo are more limited. By contrast, optical coherence tomography (OCT) may be used for label-free high-resolution imaging of the retina. In particular, spectroscopic OCT (S-OCT) enables both structural and molecular imaging of the sample by post-processing standard OCT interferograms. However, to acquire the 3D data, conventional OCT hardware requires extensive scanning. This significantly limits S-OCT in detecting small retinal amyloid plaques because of rapid eye motion. As such, there remains need for blur-free 3D imaging of retinal Aβ and pTau in vivo.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows a schematic illustration of Alzheimer's pathology across retinal cell layers in AD patients and normal control patients (CTRL).

FIGS. 2A-2I show retinal Aβ42 deposits in MCI and AD patients correlate with brain Aβ-plaque burden. FIG. 2A shows Brain histology from an AD patient displays Aβ42 plaques. FIGS. 2B-2D show retinal flatmounts histology from a subject with normal cognition (NC) and AD patients, stained with anti-Aβ42 mAb (12F4). Retinal Aβ plaques are similar in morphology to brain plaques. Scale bar: 20 μm. c′ shows high-magnification images of mature retinal Aβ plaques. Scale bar: 10 μm. FIG. 2D shows Vascular-associated retinal Aβ plaques. FIG. 2E shows marked increase of Aβ42 plaques measured in retinal flatmounts of AD patients (n=8) vs. sex-/age-matched NC control subjects (n=7). FIG. 2F-2I show retinal cross-sections. FIG. 2F shows a strip from an AD patient exhibiting Aβ42 pathology (brown) across cell layers and topographical regions. FIG. 2G shows Fluorescent tile image showing Aβ42, pTau (pSer396), and GFAP+ astrogliosis in AD retina. FIGS. 2H-I show retinal Aβ42 and vascular Aβ40 deposits identified early in MCI and AD patients.

FIGS. 3A-3B show hyperspectral imaging of Aβ and pTau deposits respectively on postmortem retinal cross sections of AD patients. From left to right, unstained hyperspectral intensity images, spectra at arrow-pointed locations, and DAB labeled images. Scale bar, 50 μm.

FIGS. 4A-4B show spectral signatures of Aβ and pTau in the human retina confirmed by combined fluorescence staining specific for Aβ42 and pS396 Tau. FIGS. 4A and 4B are two different fields of view. From left to right, unstained hyperspectral intensity images, spectra at arrow-pointed locations, fluorescence labeled Aβ42 and pS396 Tau images, and merged fluorescence images. Scale bar, 50 μm.

FIG. 5A shows a high-level schematic of operating principle of image mapping spectrometry coupled to a retinal imaging system, according to an embodiment of the disclosure.

FIG. 5B shows a high-level schematic of a snapshot spectroscopic optical coherence tomography (snapshot S-OCT) system, according to an embodiment of the disclosure.

FIG. 6 shows combination of multiple low-resolution image mapping spectrometers (IMS's) through beam splitting, according to an embodiment of the disclosure.

FIGS. 7A-7D show image processing pipeline to generate spectrally resolved volumetric images, according to an embodiment of the disclosure.

FIG. 8A shows a flowchart of steps taken to prepare training data for one or more GAN models.

FIG. 8B shows a flowchart of the training of GAN models to transform HSI models into stained images.

FIG. 8C shows a flowchart of the operation of the trained GAN models of FIG. 10B.

FIG. 9A shows the results of a GAN model trained to transform an HSI image of an Aβ deposit into an immunofluorescent-stained image of the Aβ deposit.

FIG. 9B shows the results of a GAN model trained to transform an HSI image of a pTau deposit into an immunofluorescent-stained image of the pTau deposit.

FIG. 9C shows the results of a GAN model trained to transform an HSI image of an Aβ deposit into a DAB-stained image of the Aβ deposit.

FIG. 9D shows the results of a GAN model trained to transform an HSI image of an pTau deposit into a DAB-stained image of the pTau deposit.

FIG. 10A shows a plot of structural similarity index values for the GAN models of FIGS. 9A-9D.

FIG. 10B shows a plot of peak signal-to-noise ratio values for the GAN models of FIGS. 9A-9D.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art may recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

### Overview

Alzheimer's disease (AD) and associated dementia are estimated to afflict 50 million people worldwide, a number projected to triple by year 2050. This age-dependent epidemic is a major concern for the aging population, with an incidence that rises sharply after 65 years of age, affecting roughly 50% of individuals aged 85 and older. While currently there is no cure, with early diagnosis, the progression of the disease may be slowed.

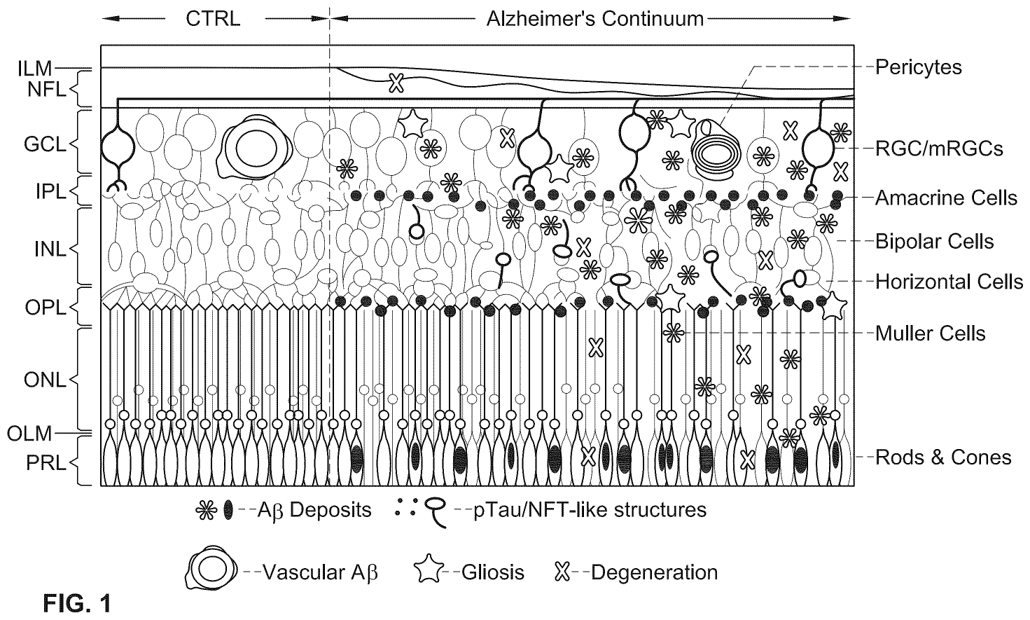

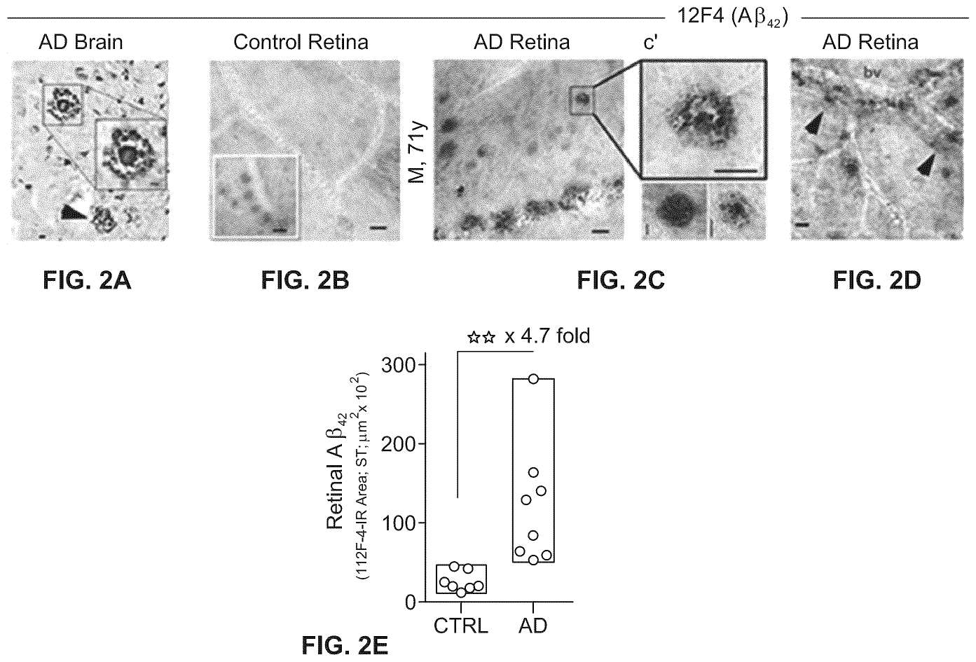

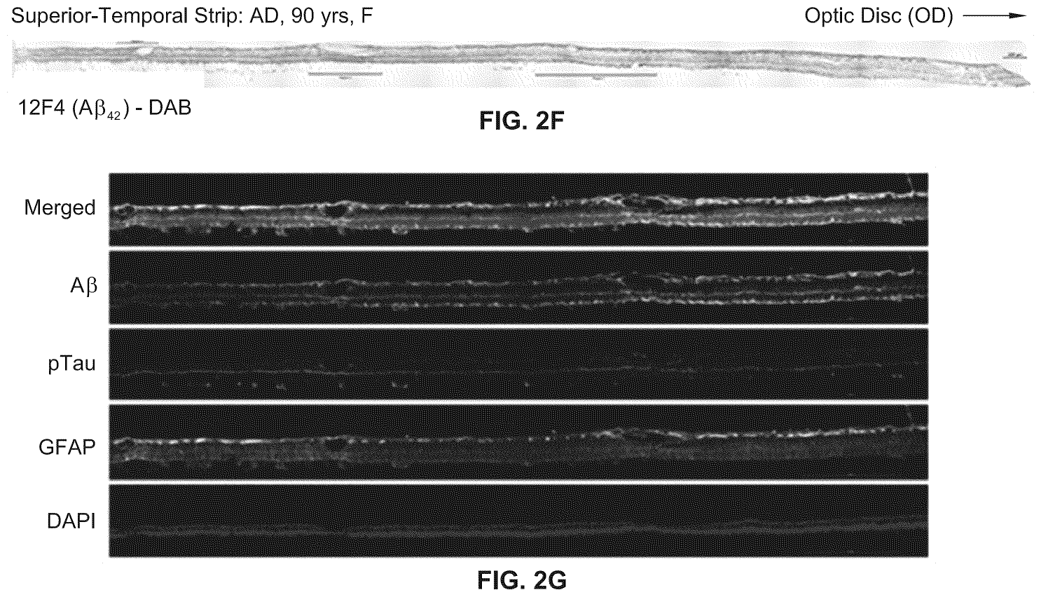

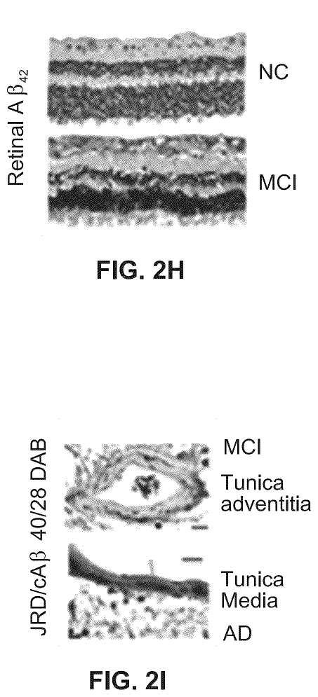

Although AD has been historically perceived as a brain disorder, recent studies indicate that AD also manifests in the eye with mounting evidence of abnormalities in the retina, a sensory extension of the brain. FIG. 1 shows Alzheimer's pathology in AD patient compared to non-AD control patients (CTRL) across retinal layers. Particularly, the hallmark pathological signs of AD, amyloid β-protein (Aβ) and neurofibrillary tangles (NFTs) comprised of hyperphosphorylated pTau protein, which have long been described in the brain, have also been identified in the retina. There is a growing number of reports that Aβ deposits and pTau were discovered in the retinas of AD patients at various stages, in stark contrast to non-AD controls. FIGS. 2A-2I show retinal Aβ₄₂ deposits in AD and MCI patients correlating with brain Aβ plaque burden.

In particular, examination of additional postmortem retinas from n=37 AD patients and controls revealed that retinal Aβ-plaque pathology in these patients was similar to brain plaques and in stark contrast to lesser pathology found in the retina of age- and sex-matched cognitively normal individuals. Perivascular and vascular Aβ deposits were also identified in retinas from MCI and AD patients (FIGS. 2A-2D). Quantification of retinal Aβ₄₂ deposits validated a substantial increase in AD patients vs. controls (FIG. 2E). Subsequent histological and in vivo imaging studies confirmed the original results and further identified pTau, characterized retinal plaque subtypes, as well as demonstrated associated inflammation and neuronal degeneration. Further, mapping of AD pathology in retinal cross sections from predefined geometrical regions in a larger MCI and AD patient cohort (n=43) uncovered the spatial and cell layer distribution of retinal Aβ₄₂, vascular Aβ₄₀ deposits, pTau (pS396), and gliosis, with significant increases of retinal Aβ₄₂ deposits in MCI/AD patients compared to NC and a strong correlation with brain Aβ plaque pathology (FIGS. 2F-2I).

The evidence of Aβ and pTau accumulation in the retina at early stages of AD lends credence to the notion of the eye as a site for pre-symptomatic stage imaging. In particular, retinal Aβ plaques and oligomers in transgenic AD-model mice appear at the pre-symptomatic stage and prior to detection in the brain. Further, retinal pTau appear in AD. Because the retina can be imaged directly and noninvasively, detection of retinal AD pathology, particularly the early presenting Aβ and p(tau) biomarkers, may allow for large-scale screening and monitoring of at-risk populations.

Despite holding great promise for early diagnosis of AD, imaging retinal Aβ and pTau deposits is non-trivial. Because Aβ and pTau deposits have a similar visual appearance to normal tissue, conventional fundus photography provides little contrast. In some approaches, by using exogenous fluorophores, retinal Aβ and pTau may be visualized through fluorescence imaging. Yet, administrating contrast agents in humans complicates the imaging procedure, posing hindrances for screening large-scale populations. Also, to date, only curcumin, a natural fluorochrome, has been used in clinical trials to label retinal Aβ while fluorophore choices to visualize in vivo retinal pTau are more limited. Therefore, there is an unmet need to develop label-free, high-resolution imaging techniques to visualize retinal Aβ and pTau deposits for early AD screening and disease management.

The inventors herein have recognized the above-mentioned issues, and provide systems and methods to at least partially address the above disadvantages. In one example, an integrated retinal imaging system comprises a retinal fundus imaging unit; and a snapshot hyperspectral imaging unit integrated with the retinal fundus imaging unit; wherein the retinal imaging system is configured to image portions of a retina, the portions of the retina corresponding to one or more of amyloid β-protein (Aβ) and hyperphosphorylated pTau protein aggregation; and wherein the retinal imaging system is configured to acquire retinal images in a snapshot format.

Further, the inventors have identified that Aβ and pTau deposits (without any fluorophore/chromophore/contrast labelling) exhibit a significant difference from normal tissue in the 500-650 nm wavelength. Accordingly, spectrally resolved volumetric images in the 500-650 nm range can be used to identify Aβ and pTau deposits in retinal layers without any contrast/chromophores in vivo. Thus, by combining snapshot hyperspectral imaging with an in vivo retinal imaging modality, and image processing to re-build the data cube using light re-directed on a detector of the hyperspectral imager from an image mapper (comprising plurality of facets), the desired spectrally-resolved 3D sample structure may be obtained, based on which Aβ and pTau deposits may be identified.

Further, in some examples, Aβ and pTau deposits may be quantified in order to determine whether the subject has pathological hallmarks of AD and MCI, as well as a degree of progression of the disease.

Furthermore, in some examples, when the subject is undergoing immunomodulation treatments, reduction in Aβ and pTau deposits may be monitored over a duration of the treatment.

Further still, in some examples, the integrated retinal imaging system may be utilized to generate datasets, including training and testing datasets, for training a deep learning algorithm for Aβ and pTau deposit classification in an efficient manner.

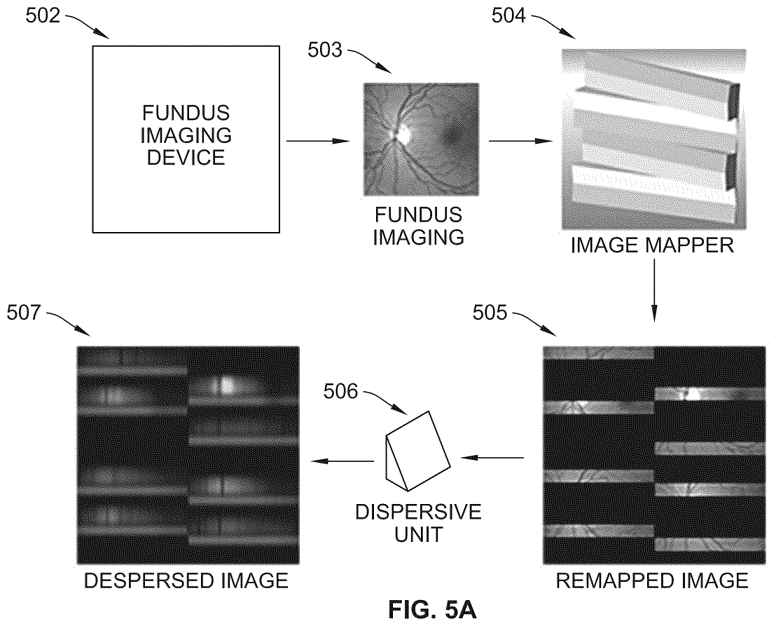

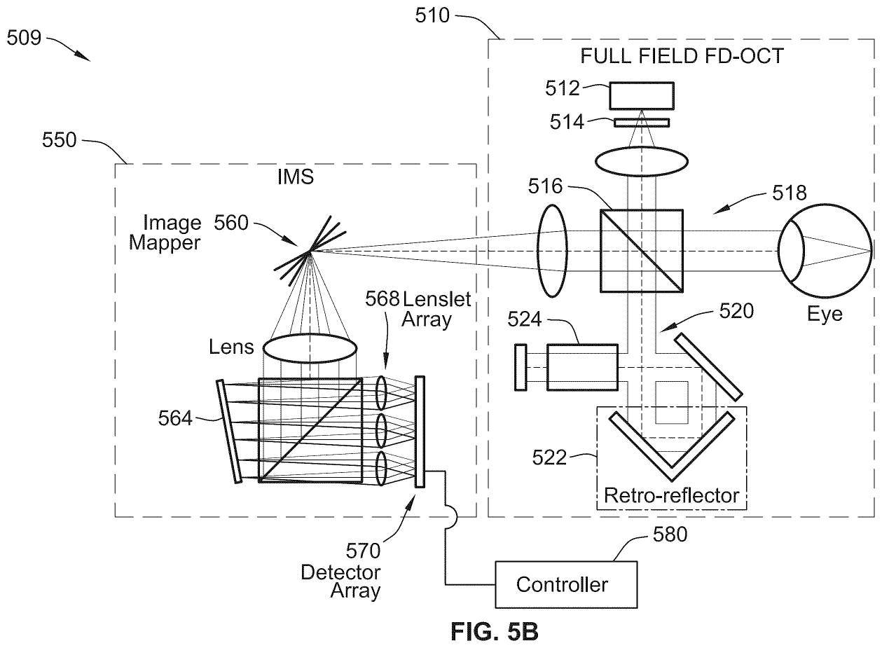

Referring to FIG. 5A, in one example, the retinal fundus imaging unit 502 may be used to generate a fundus image 503. The retinal fundus imaging unit 502 can be an optical coherence tomography (OCT) system. In particular, spectroscopic OCT (S-OCT) may be used to acquire in vivo retinal images 503 without use of contrast. OCT is noninvasive, and has 3D imaging capability. While conventional OCT provides the morphological and layer information of the sample, spectroscopic OCT (S-OCT) expands the functionality of OCT to both structural and molecular imaging. Through post-processing, S-OCT uses the interferograms generated by OCT to derive the depth-resolved spectroscopic profiles of a sample. The spectroscopic information so obtained may be used to fingerprint endogenous chromophores, endowing an OCT image with molecular contrast. S-OCT may be performed by applying time-frequency transformation (TFT) to the spectral interferograms obtained by a Fourier-domain OCT (FD-OCT) system.

In one example, a short-time Fourier transform (STFT), which is essentially a windowed Fourier transform:

$$S ( k , z ) = STFT [ I ( k ) ] = \int I ( {k}^{′} ) w ( {k}^{′} - k ; Δ k ) {e}^{- {ik}^{′} z} {dk}^{′} , \qquad ( 1 )$$

where k is the wavenumber, and w(k) is a window function with a spectral bandwidth of Δk. The window function w(k) slides incrementally into a spectral interferogram I(k), and the segments confined by the window are Fourier-transformed to generate the spectrally resolved depth profile S(k, z). Therefore, S-OCT can provide not only a depth-resolved structure of a sample, but also spectroscopic information for a specific depth within the bandwidth of the light source.

However, in order to acquire real-time 3D imaging for S-OCT, previous conventional OCT hardware requires extensive scanning to obtain full-field spectral interferograms I(x, y, k) (x, y, transverse spatial coordinates). The scanning mechanism causes a trade-off between the number of photons the instrument can collect and the frame acquisition rate, limiting the system in imaging fast dynamics. In retinal imaging, because of the rapid movement of the eye, the slow acquisition speed may result in severe motion artifacts, blurring the image and degrading the resolution. This is particularly problematic for detecting retinal Aβ and pTau aggregates because of their small micrometer sizes. Although the imaging speed of FD-OCT can be significantly increased in the cross-sectional-scan (B-scan) mode, the limited lateral region imaged could lead to the missing detection of retinal amyloid plaques, which frequently appear in scattered clusters.

The inventors have identified the above disadvantages of applying scanning to acquire S-OCT images. In one example, in order to acquire 3D molecular imaging of retinal AD biomarkers, such as Aβ and pTau aggregates, full-field spectral interferogram may be acquired in a snapshot format.

In one example, in order to enable snapshot imaging, a snapshot hyperspectral imager, such as image mapping spectrometry (IMS) may be used. The IMS may replace the camera in a digital imaging system, thereby adding high-speed snapshot spectrum acquisition capability to S-OCT to maximize the collection speed.

Conventional spectral imaging devices acquire data through scanning, either in the spatial domain (as in confocal laser scanning microscopes) or in the spectral domain (as in filtered cameras). Because scanning instruments cannot collect light from all elements of the dataset in parallel, there is a loss of light throughput by a factor of Nx×Ny when performing scanning in the spatial domain over Nx×Ny spatial locations, or by a factor of Nλ when carrying out scanning in the spectral domain measuring Nλ spectral channels.

The IMS is a parallel acquisition device that captures a hyperspectral data cube without scanning. It also allows full light throughput across the whole spectral collection range due to its snapshot operating format. The IMS uses an image mapper 504 (which in some cases is a custom-designed mirror), which comprises multiple angled facets to redirect portions of an image to different regions on a detector array that can produced a remapped image 505. By redirecting slices of the image so that there is space between slices on the detector array, a dispersive unit 506 (which may be a prism or a diffraction grating) may be used to spectrally disperse light in the direction orthogonal to the length of the image slice to generate a dispersed image 507. In this way, with a single frame acquisition from the retinal imaging unit, a spectrum from each spatial location in the image may be obtained. The original image is then reconstructed by a remapping of the pixel information.

To reflect the image zones into different directions, individual facets of the image mapper 504 have different tilt angles with respect to the two axes in the plane of the slicer. This mapping method establishes a fixed one-to-one correspondence between each voxel in the data cube (x,y,λ) (x,y, spatial coordinates; λ, wavelength) and each pixel on the camera. The position-encoded pattern on the camera simultaneously provides the spatial and spectral information within the image. Since the acquired data results directly from the object's irradiance, no reconstruction algorithm is required, and image remapping produces the image and data displays.

In this way, by integrating hyperspectral imaging system with a retinal fundus imaging unit, such as S-OCT, pathological hallmarks of AD may be detected and monitored. The integrated system may further facilitate diagnosis of other diseases involving Aβ accumulation and/or tauopathies, such as CAA, frontotemporal dementia, and age-related macular degeneration (AMD). The retinal imaging system that could detect the earliest, most specific molecular signs of AD, Aβ and pTau, in vivo could provide an early warning for individuals at risk for AD, as well as facilitate the development and assessment of new therapies based on specific molecular profile. The knowledge obtained from pre-symptomatic individuals with AD pathology may help our understanding of the genesis of AD in the retina at its very earliest roots and thereby provide optimal guidance in search of an early detection and a cure. Moreover, the noninvasive label-free imaging technique may make our approach particularly suitable for large-scale population screening in regular office-based settings, extending profound health and social benefits to our aging population.

The integrated system comprising S-OCT and IMS is referred to herein as “snapshot S-OCT”. The snapshot S-OCT is a label-free imaging modality that can detect the 3D distribution of retinal Aβ and pTau deposits in vivo. The snapshot S-OCT system may allow high-resolution spectroscopic imaging of the retina in 3D in a single camera exposure. Further, deep learning methods may be used in classifying retinal Aβ and pTau within pre-defined topographical and cell layers in retina and brain of neuropathologically well-characterized human donor tissues.

The parallel acquisition scheme of snapshot S-OCT may also be used for generating the training data for deep learning. Furthermore, the application of snapshot S-OCT in detecting retinal AD biomarkers (e.g., the presence of Aβ and/or pTau deposits in a patient's retina) provides blur-free imaging which makes the snapshot S-OCT system particularly suitable for detecting small retinal and pTau deposits, which is not possible with previous scanning-based approaches. The label-free 3D molecular imaging and snapshot advantage uniquely positions snapshot S-OCT to address the leading challenges in live imaging of retinal AD pathological hallmarks.

An example snapshot hyperspectral imaging of in vivo retina by image mapping spectrometry (IMS) is shown by Gao, L. et al. in Snapshot hyperspectral retinal camera with the Image Mapping Spectrometer (IMS). Biomed Opt Express 3, 48-54 (2012), which is incorporated herein by reference in its entirety.

In one example approach, snapshot optical coherence microscopy by image mapping spectrometry was performed to image retinal samples. This approach is shown by Iyer, R. R. et al. in Full-field spectral-domain optical interferometry for snapshot three-dimensional microscopy. Biomed Opt Express 11, 5903-5919 (2020). PMCID: PMC7587259, which is incorporated by reference in its entirety. Therein, the prepared sample is illuminated by a broadband LED. The back reflected light beams from the sample and reference arms are combined by a beam splitter, and full-field spectral interferograms are measured by the IMS camera. Through a single snapshot, the system can measure a 3D volume with a transverse resolution of 0.8 μm, an axial resolution of 1.4 μm, and a sensitivity of up to 80 dB. The OCM system performance was demonstrated by imaging mouse mesangial cells cultured densely on a flat surface. Due to a relatively low spectral resolution (˜7 nm) of the IMS, the depth range of the OCM is only 20 μm. By contrast, in the systems and methods described herein (that is, snapshot S-OCT system), a high-spectral-resolution (˜0.4 nm) IMS is implemented. When combining with OCT, a depth range of 200 μm may be achieved with the snapshot S-OCT system. Due to the low depth range, snapshot OCM cannot be translated to in vivo imaging of retinal cells. Further, even if adapted for in vivo imaging of retinal cells, the snapshot OCM, the low depth range does not enable detection of AD pathological hallmarks.

The inventors herein have identified the above-mentioned disadvantages of snapshot OCM, and provide systems and methods for detecting AD pathological markers, including Aβ and pTau deposits, in vivo and without the use of exogenous fluorophores, and further, with a single snapshot acquisition. In one example, an integrated system (S-OCT) may be configured to capture the specific spectral signatures of retinal Aβ and pTau deposits and identify their 3D distributions within a single camera exposure, eliminating the motion artifact and thereby enabling a high resolution. The snapshot S-OCT may provide improved accuracy in detecting various types of retinal Aβ and pTau assemblies as compared to fluorescence imaging, and moreover, without the need for contrast agents.

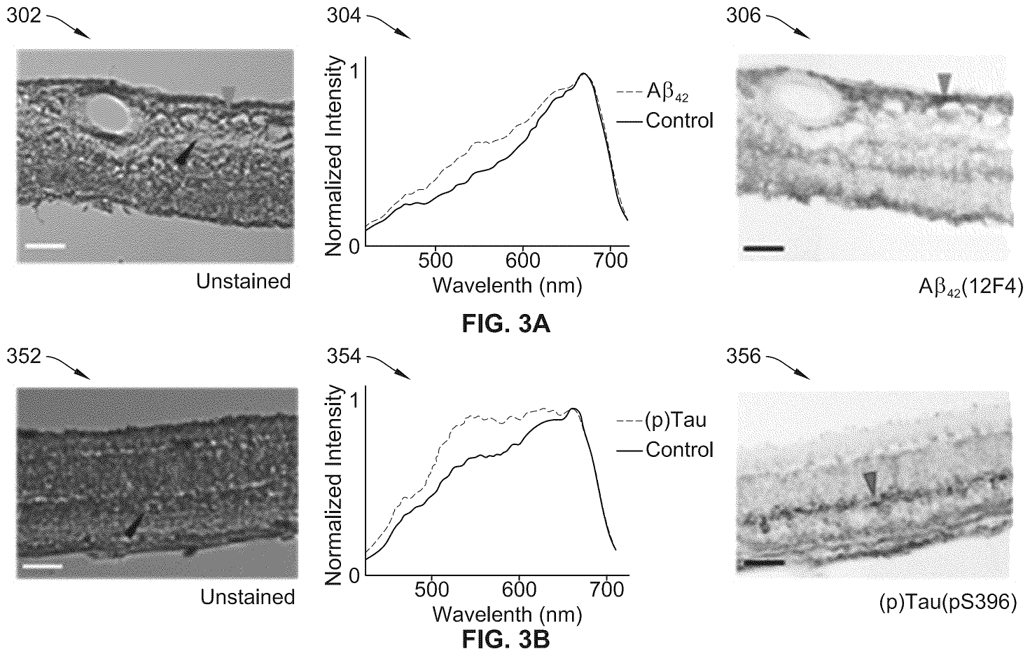

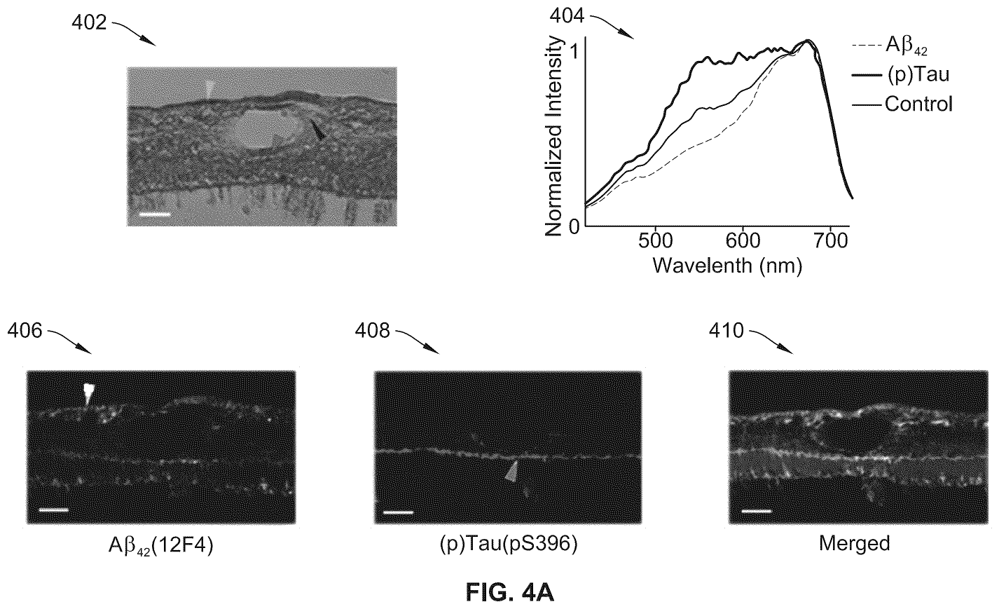

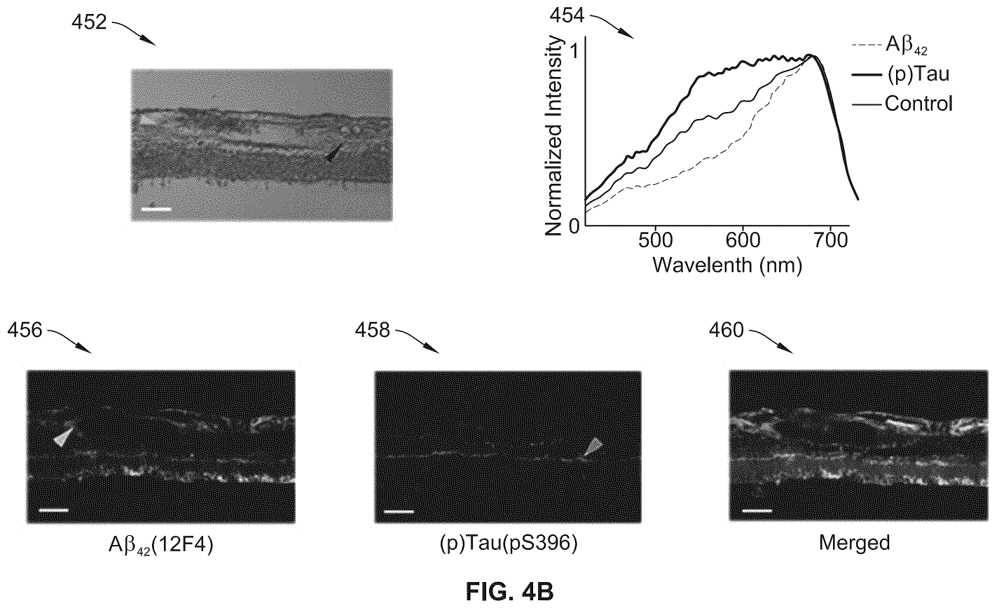

In one example, the inventors have, for the first time, identified that 1) AD pathological markers, Aβ and pTau deposits, each have unique spectral signatures compared to normal tissues, and 2) the wavelength range where the spectra of Aβ and pTau deposits exhibit a significant difference from normal tissue is in the 500-650 nm wavelength range. The signature spectra of Aβ and pTau deposits are further discussed below with respect to FIGS. 3A, 3B, 4A, and 4B.

Hyperspectral Imaging of Aβ and pTau Deposits in Human Postmortem Retinal Cross-Sections

In a preliminary study, unstained postmortem retinal cross sections from AD patients (N=5) were imaged on a microscope. The samples were illuminated using a broadband halogen lamp, filtered the image using a liquid-crystal tunable filter (Thorlabs, 10 nm bandwidth), and measured spectrum at each pixel. Image 302 in FIG. 3A shows an example unstained hyperspectral intensity image (also referred to herein as an HSI image) of Aβ deposits, and image 352 in FIG. 3B shows an example unstained hyperspectral intensity image of pTau deposits. Image 402 in FIG. 4A shows an example unstained HSI image of Aβ and pTau deposits from a first fields of view, while image 452 in FIG. 4B shows an example unstained HSI image of Aβ and pTau deposits from a second field of view. Plot 304 in FIG. 3A shows the spectra of the Aβ deposits in image 302, and plot 354 in FIG. 3B shows the spectra of pTau deposits in image 352. Plots 404 and 454 in FIGS. 4A and 4B show the spectra of the Aβ and pTau deposits in images 402 and 452 from the two different fields of view. The spectra of the Aβ deposits and the pTau deposits were identified by averaging the pixel spectra in the regions of the images 302, 352, 402, and 452 where the deposits appeared.

Illumination regions were guided by immunostaining specific to Aβ₄₂ and pS396-Tau in an adjacent retinal cross section. Importantly, for validation of the hyperspectral signatures, the same imaged samples were immunostained with the following specific antibody combination: mouse anti-Aβ 1-42 monoclonal antibody, 12F4 (1:500, BioLegend #805502) and rabbit anti-pTau polyclonal antibody, pSer396 (1:2500, AS-54977). Retinal cross-sections were either imaged by peroxidase-based 3,3′-Diaminobenzidine (DAB) labeling, each antibody was single stained, or fluorescently labeled via incubation with secondary antibodies (1:200; Cy5 conjugated donkey anti mouse and Cy3 conjugated donkey anti rabbit, Jackson ImmunoResearch Laboratories). Image 306 in FIG. 3A is a DAB-stained image of the Aβ deposits, and image 356 in FIG. 3B is a DAB-stained image of the pTau deposits. Image 406 in FIG. 4A is a fluorescence-stained image of the Aβ deposit from the first field of view, image 408 in FIG. 4A is a fluoresence-stained image of the pTau deposit from the first field of view, and image 410 in FIG. 4A is a merged fluorescence-stained image of the Aβ deposit and the pTau deposit from the first field of view. Image 456 in FIG. 4B is a fluorescence-stained image of the Aβ deposit from the second field of view, image 458 in FIG. 4B is a fluoresence-stained image of the pTau deposit from the second field of view, and image 460 in FIG. 4B is a merged fluorescence-stained image of the Aβ deposit and the pTau deposit from the second field of view.

By registering the hyperspectral images (302 and 352) with the immuno-labeled image (306 and 356), enriched areas of Aβ₄₂ and pTau can be located in the hyperspectral images. The spectral signatures of Aβ₄₂, pTau, and normal tissue (control) can be extracted from the hyperspectral measurement, as shown in plots 304 and 354. In some cases, the spectra of Aβ and pTau deposits exhibit a significant difference from normal tissue in the 500-650 nm wavelength range. More importantly, for the first time, the spectral signature of retinal pTau deposits was identified. This observation was confirmed by imaging multiple fields of view/patients, and the results are consistent, as shown by the curves in plot 304 and 343.

### The Snapshot S-OCT System

Turning now to FIG. 5B, it shows an example snapshot S-OCT system 509. The snapshot S-OCT system 509 comprises a full-field FD-OCT system 510 and an IMS 550.

The full-field FD-OCT system 510 includes a light source 512. In one example, the light source may be a flash lamp (duration, 4 μs; Hamamatsu LF2 flashlight source). The light emitted from the light source may be filtered with a visible-light bandpass filter 514 (central wavelength, 550 nm; bandwidth, 100 nm) to illuminate the retina. In some embodiments, the light source may be a visible light source emitting light in the visible range between 400 nm and 700 nm. In some other embodiments, the light source may be a broadband light emitting diode (LED) source having a wavelength range in the visible spectrum. In further embodiments, near infrared (near IR) wavelengths may be used.

The full-field FD-OCT system 510 may employ a static interferometer setup. Light emitted from the filtered light source is divided between sample and reference arms by a non-polarization beamsplitter 516 into a reference beam channel 520 and a sample channel 518 that directs light to a sample (that is, the eye). The reference beam channel 520 passes a retroreflector 522 mounted on an automated translation stage for controlling the reference optical path length and a water vial 524 for dispersion balancing the subject's eye for in vivo imaging. The photons reflected from the retina interfere with the reference beam, forming spectral interferograms in the output intermediate image.

The output intermediate image is then sampled by an image mapper 560 in the IMS 550. In one example, to achieve a target of 400×400 (x,y) spatial pixels, the mapper may comprise 400 total facets (that is, number of facets is 400), each 25 μm wide and 10 mm in length. On the mapper, a total of 255 assorted 2D tilt angles (a combination of 17 x tilts and 15 y tilts) may be fabricated, enabling hyperspectral imaging of 255 spectral bands. The parameters of the image mapper are shown in Table 1 below.

TABLE 1
Parameters of the image mapper.

| | |
|---|---|
| # of mirror facets | 400 |
| Mirror facet length | 10 mm |
| Mirror facet width | 25 μm |
| Mirror facet x tilts | ±N × 0.0167 radians |
| | (N = 0:8) |
| Mirror facet y tilts | ±M × 0.0167 radians |
| | (M = 0:7) |

In one example, the image mapper may be fabricated using a diamond ruling technique. For mass production the fabrication cost of image mappers can be minimized by employing injection molding techniques.

At the image mapper, the sample's imaged point spread function (PSF) may be matched to the width of mirror facet, resulting in an effective NA of 0.015. The light rays reflected from different mirror facets are collected by an objective lens (e.g., focal length, 90 mm; Olympus MVX PLAPO1×) and enter the corresponding pupils at the back aperture of the lens. The angular separation distance between adjacent pupils is 0.033 radians, which is greater than twice of the NA (0.03) at the image mapper, thereby eliminating the crosstalk between pupils.

The light is may then be spectrally dispersed by a high-resolution ruled grating 564 (e.g., 1200 grooves/mm; blaze wavelength, 550 nm; Littrow configuration; Optometrics) and reimaged by an array of lenslets 568 (e.g., 17×15; focal length, 22.5 mm; diameter, 2.5 mm). The full-field spectral interferograms are then measured a large-format detector array 570 (e.g., 8176×6132 pixels; pixel size, 6 μm; E7 camera, MegaVision) within a single exposure. Since the magnification from the image mapper 560 to the detector array 570 is 0.25, the image associated with each lenslet of the lenslet array 568 is 2.5×2.5 mm² in size, sampled by 400×400 camera pixels. The spacing created for spectral dispersion between two adjacent image slices is 1.6 mm and sampled by ˜250 camera pixels. Given 100 nm spectral bandwidth, the resultant spectral resolution is approximately 0.4 nm. The system may be calibrated as described in Bedard, N. et al., in Image mapping spectrometry: calibration and characterization. Optical Engineering 51 (2012), which is incorporated herein by reference in its entirety.

In some examples, the number of facets may be based on a desired depth range for imaging portion of retina where Aβ and pTau aggregates may be found. In various embodiments, the number of facets may be any number between 100 and 600. Further, the grating parameters and the lenslet array parameters may be configured based on the number of facets.

The detector array 570 may be communicatively coupled (e.g., via a wired and/or wireless connection) to a controller 580 and image data from the detector array 570 may be processed via the controller 580 and displayed in real-time or near real-time via a display portion of a user interface communicatively coupled to the controller.

The controller 580 may include at least one processor (CPU) and memory such as read-only memory ROM and/or random-access memory RAM, which comprise computer-readable media that may be operatively coupled to the processor. Thus, one or more of ROM and RAM may include system instructions that, when executed by the processor performs one or more of the operations described herein, such as the process flow of subsequent figures. Processor can receive one or more input signals from various sensory components and can output one or more control signals to the various control components described herein via input/output (I/O) interface. In some examples, one or more of the various components of controller 580 can communicate via a data bus. The present example shows an example configuration of the controller 580, it will be appreciated that the controller 580 may be implemented with other configurations.

The controller 580 may provide synchronized control of all opto-mechanical components within the system 509. For example, the controller 580 may rapidly perform optical alignment between various components and enable simultaneous image acquisition with a plurality of detectors (or cameras) within the detector array 570.

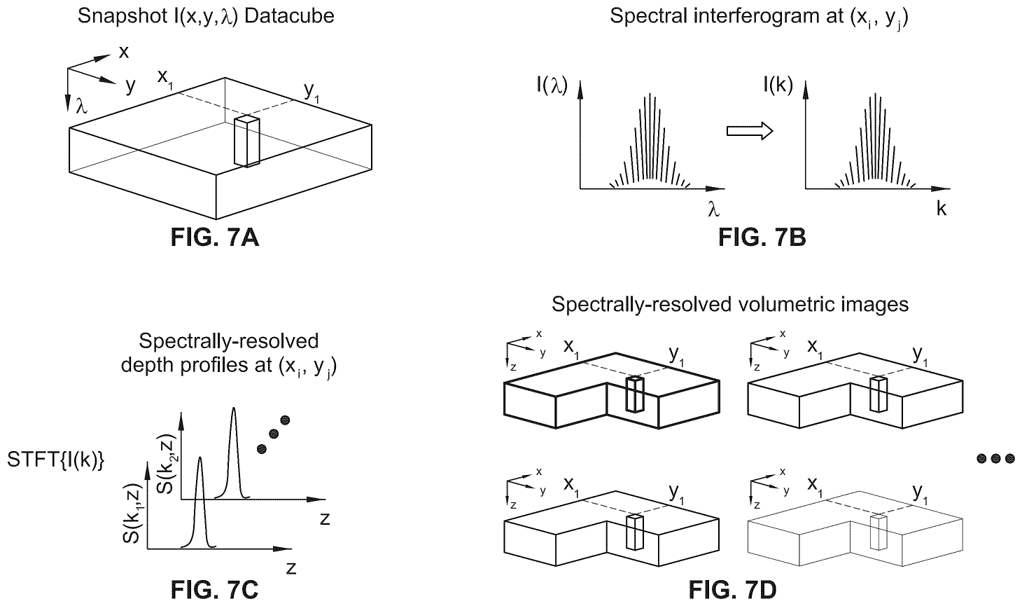

The controller 580 may perform image post-processing according to instructions stored in non-transitory memory, such as ROM and RAM. For example, the controller 580 may perform one or more of data cube re-mapping and generate spectrally resolved 3D structures on raw data acquired via the detector array. Further, the controller 580 may perform image analysis on post-processed images. For example, the image analysis may be performed according a trained deep learning algorithm as described below, among other image analysis methods. Details of image processing pipeline is further described below at FIGS. 7A-7D.

In some examples, the controller may store a trained deep learning algorithm for classifying Aβ and pTau pathological hallmarks of AD.

In some other examples, the controller may be configured to generate training and validation data sets for training the deep learning algorithm for classifying Aβ and pTau deposits.

FIGS. 7A-7D show an example image processing pipeline. Data processing
includes calibration of the image-sliced data, to rebuild the I (x, y, λ) data cube following re-direction at the image mapper. Re-mapping algorithms for the snapshot S-OCT system may be used and may generate the corresponding re-mapping parameters for the new mapper to be fabricated. Once the I(x, y, λ) data cube is correctly assembled, S-OCT image processing steps may be applied to generate the desired spectrally-resolved 3D sample structure, as illustrated in FIGS. 7A-7D. Each location on the sample surface is associated with a separate interferogram, I(xᵢ, yⱼ, λ), with i=1-400 and j=1-400 corresponding to pixels in x- and y-dimensions (FIG. 7A). Conversion of interferograms to A-lines includes re-sampling of raw data from the wavelength 1 to the wavenumber k domain (k=2π/λ) (FIG. 7B), followed by short-time Fourier transformation to the spectrally resolved z-space (FIG. 7C). Repeating the procedure above at each transverse location may yield spectrally resolved volumetric images/(x, y, z, k) (FIG. 7D).

For in vivo imaging, an important factor to account for includes the spectral transmission of the ocular lens, which is generally unpredictable in an older population. Because previous studies show that both retinal Aβ and pTau are primarily distributed in peripheral superior and inferior retinal quadrants for the same subject, first, a control area may be imaged in the nasal retinal quadrant where no blood vessels are visible in the field of view. Then, the average spectrum of normal retinal substance in this control area may be calculated as

$${I}_{0} ( k ) = \frac{1}{{N}_{x} {N}_{y} {N}_{z}} {\sum }_{l , m , n}^{{N}_{x} , {N}_{y} , {N}_{z}} {I}_{0} ( l , m , n , k ) ,$$

where l, m, n are image voxel indices along x, y, z axis, respectively, and Nₓ, N_{y}, N_{z} are the total numbers of image voxels along x, y, z axis, respectively. For the following measurements, the relative spectrally resolved optical densities may be computed as:

$$OD ( x , y , z , λ ) = log [ {I}_{0} ( k ) / I ( x , y , z , k ) ] . \qquad ( 2 )$$

Because both I₀(k) and I(x, y, z, k) are measured on the same subject, the effect of the ocular lens on the transmitted spectrum may be canceled out.

In some examples, detection of retinal Aβ and pTau from OD(x, y, z, k) may be performed based on a deep-learning HSI classification method. Briefly, spectral and spatial information may be combined to construct spectral-spatial features for each image pixel at a given depth layer. The neighboring region of a center pixel includes eight en-face rays in a 45-degree interval. The pixels along the ray are extended around the pixel with a radius (e.g., ten pixels). The pixels may be flattened along the ray into one vector and use it as the spatial feature of the center pixel. As each pixel along the ray also has multiple spectral bands. Further, all the bands may be flattened into one long vector. The resultant spatial-spectral dataset may be fed into a deep neural network, and latent representations can be learned using stacked auto-encoders (SAE). To integrate the layers of neural networks and perform classification based on the feature learned, the algorithm tunes the whole network with a multinomial logistic regression classifier. Backpropagation may be used to adjust the network weights in an end-to-end fashion.

Measured by the IMS, the hyperspectral data cube voxels are one-to-one mapped to the detector array pixels. Therefore, the system design faces a trade-off between the spatial and spectral samplings—the product of spatial and spectral samplings (Nₓ×N_{y}×N_{λ}) cannot exceed the number of camera pixels (8176×6132). Also, the spectral sampling determines the depth range zₘₐₓ in FD-OCT through the relation zₘₐₓ=N_{λ}λ₀²/(4λ_{full}), where λ₀ is the central wavelength of light, and λ_{full} is the full spectral bandwidth of the camera. Given an illumination source λ₀=550 nm, λ_{full}=100 nm in the proposed system, the maximum depth range zₘₐₓ is 0.8N_{λ} μm. To cover several retinal layers (NFL/GCL and OPL) where Aβ and p-Tau typically cluster, N_{λ}=250 may be chosen, yielding a ˜200 μm depth range. Accordingly, to balance the lateral samplings for this desired depth range, Nₓ=N_{y}=400 may be set. Given a 10° field of view, the resultant lateral resolution approximates 7 μm, which has been demonstrated enough in resolving Aβ and pTau accumulates. On the other hand, in S-OCT, when using STFT with a Gaussian window function, the resultant axial resolution, Δz, and spectral resolution, Δλ, are limited by the uncertain principle, i.e., ΔzΔλ=λ₀²/2. Therefore, there is a trade-off between the axial resolution and spectral resolution. To resolve the retinal layers where Aβ and pTau primarily aggregate (NFL, GCL, and OPL), a window function that leads to Δz˜15 μm may be chosen. The resultant spectral resolution may be ˜10 nm, which may be sufficient to detect the spectral signatures of Aβ and pTau based on our preliminary data. The system parameters are summarized in Table 2 below.

TABLE 2
Imaging parameters of the proposed snapshot S-OCT system

| | |
|---|---|
| 3D image volume (x, y, z) | 400 × 400 × 13 |
| Lateral resolution | 7 μm |
| Depth range | 200 μm |
| Depth resolution | 15 μm |
| Spectral resolution | 10 nm |

Measured by the IMS, the hyperspectral data cube voxels are one-to-one mapped to the detector array pixels. Therefore, the system design faces a trade-off between the spatial and spectral samplings—the product of spatial and spectral samplings (Nₓ×N_{y}×N_{λ}) cannot exceed the number of camera pixels (8176×6132). Also, the spectral sampling determines the depth range zₘₐₓ in FD-OCT through the relation zₘₐₓ=N_{λ}λ₀²/(4λ_{full}), where λ₀ is the central wavelength of light, and λ_{full} is the full spectral bandwidth of the camera. Given an illumination source λ₀=550 nm, λ_{full}=100 nm in the proposed system, the maximum depth range zₘₐₓ is 0.8N_{λ} μm. To cover several retinal layers (NFL/GCL and OPL) where Aβ and p-Tau typically cluster, N_{λ}=250 was chosen, yielding a ˜200 μm depth range. Accordingly, to balance the lateral samplings for this desired depth range, Nₓ and N_{y} were both set equal to 400. Given a 10° field of view, the resultant lateral resolution approximates 7 μm. On the other hand, in S-OCT, when using STFT with a Gaussian window function, the resultant axial resolution, Δz, and spectral resolution, Δλ, are limited by the uncertain principle, i.e., ΔzΔλ=λ₀²/2. Therefore, there is a trade-off between the axial resolution and spectral resolution. To resolve the retinal layers where Aβ and pTau primarily aggregate (NFL, GCL, and OPL), a window function that leads to Δz˜15 μm was chosen. The resultant spectral resolution is ˜10 nm, which is sufficient to detect the spectral signatures of Aβ and pTau.

The signal-to-noise ratio (SNR) that can be expected with the proposed method was estimated using the framework developed by Hillmann et al. in Aberration-free volumetric high-speed imaging of in vivo retina. Sci Rep-Uk 6 (2016), which is incorporated by reference in its entirety. Therein they presented a full-field FD-OCT system that generates spectral interferograms by tuning a narrow linewidth source across a bandwidth of 50 nm. By contrast, the proposed method essentially captures the interference at each wavelength simultaneously, by using a broadband light source and a single, large-format camera frame. With only 0.085 μJ of illumination energy per wavelength distributed over 0.3 megapixels, Hillmann et al. predicted an SNR of 75 dB. Provided the system is shot-noise limited, the snapshot S-OCT system may achieve the same SNR over our 50 megapixels by delivering 15 μJ illumination energy to the retina, which is within the capability of the light source (˜10 mJ per flash, at 550 nm, 100 nm bandwidth). Herein 10% light coupling efficiency may be assumed between the lamp and system to account for the spatial incoherence of the illumination source. Also, the illumination fluence at the retina (1.5 mJ/cm²) is well below the ANSI laser safety standard (˜80 mJ/cm²).

In one example, a method for detecting Aβ and/or pTau deposits in vivo and without using contrast agents comprises, generating spectrally resolved volumetric data via the snapshot S-OCT system; identifying a desired spectral range; and identifying Aβ and/or pTau deposits based on deviation of spectral signature from normal at the desired spectral range; wherein the deviation of spectral signature is based on irradiance; and wherein the desired spectral range is between 500 nm and 650 nm.

### System Characterization and Validation

To characterize the 3D imaging performance, a tissue phantom consisting of TiO₂ scatterers suspended in silicone with an average diameter of 1 μm may be imaged. The phantom may be placed at the back focal plane of a singlet lens with a focal length of 20 mm, mimicking the crystalline lens of the eye. Additionally, the space between the lens and phantom may be filled with a clear gelatin gel to mimic the vitreous body. The primary outcome of the measurement may include the lateral and axial resolutions, signal-to-noise ratio, field of view, and depth range. To provide a gold standard, the phantom may be directly imaged using a reflectance laser scanning confocal microscope (Leica SP8).

To evaluate the spectral imaging performance, a phantom that comprises polyethylene microspheres of assorted colors (green, yellow, and orange; diameter, 10 μm; Cospheric) uniformly mixed and sealed in a gel may be imaged. The spectra of each microsphere may be measured using the proposed approach. To provide the ground truth, another thin layer of microspheres may be prepared in a microscopic slide, the sample may be back illuminated with the same light source, and directly image the microspheres using an inverted microscope equipped with a calibrated liquid crystal tunable filter (Thorlabs). The spectral accuracy may be quantified by calculating the RMSE of the spectral difference between the normalized spectra S_{H}(λ) measured by the proposed method and ground truth S₀(λ), RMSE=\sqrt{Σ_{λ}[S_{H}(λ)−S₀(λ)]²/M}, where M is the total number of spectral channels, where mean RMSEs for all three colored microspheres may be no greater than 5%.

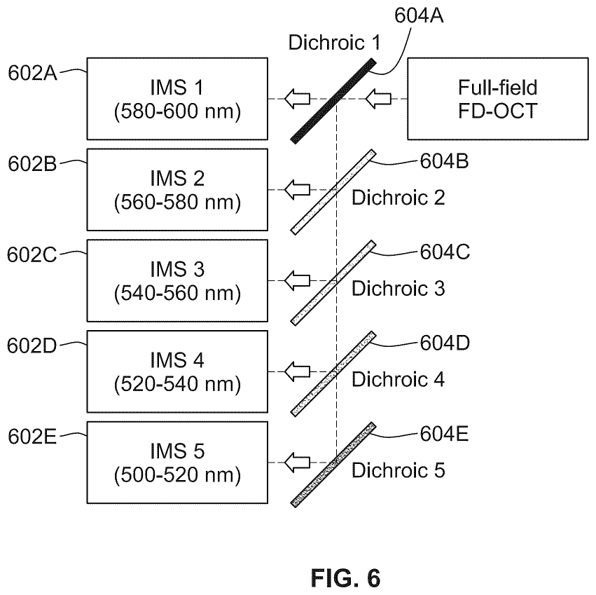

In some embodiments, using multiple low-resolution IMS's, each measuring a separate spectral range, may be used. In particular, several duplicated low-resolution IMS's may be used replacing their spectral dispersion units with ruled gratings. Next, their optical paths may be combined using dichroic filters with a descending order of their cut-off wavelengths. The schematic is shown in FIG. 6, which includes five IMS's 602A-602E, and five dichroic filters 604A-604E. Each IMS 602A-602E provides 50 spectral samplings in the correspondent spectral band, allowing a total of 250 spectral channels in the wavelength range of the filtered light source. The resultant system may have a similar spectral resolution (0.4 nm) as that offered by the high-spectral-resolution IMS.

Validation of Snapshot S-OCT in Detecting Aβ and pTau Pathology in Postmortem Retinal Tissues from AD and MCI Patients

The snapshot S-OCT system may be evaluated on unstained postmortem retinal and brain tissues in comparison to immuno-labeled tissues. Further, the distribution of retinal Aβ₄₂ and Aβ₄₀ deposits, vascular Aβ, and pTau forms specific to AD may be quantified with the snapshot S-OCT system and the results may be compared with the quantitative immunohistochemistry (IHC) data. In this way, snapshot S-OCT may be validated as a high-resolution, label-free alternative to fluorescence approaches in detecting various retinal AD biomarkers.

Paired samples of postmortem eyes and brains may be obtained, and Flatmounts and cross sections of neurosensory retinas and brains may be prepared from new and previously collected human donor tissues. The diagnosis may be confirmed by postmortem neuropathology plus clinical records on antemortem cognitive status. Human subject's demographics include sex, age, and race/ethnicity. Subjects may be matched for age (mean ˜80 years-old) and sex (females and males at equal numbers). Tissue isolation, processing and immunostaining may be performed.

### Snapshot S-OCT Imaging and Histological Validation.

To image the retinal flatmount using the proposed system, a 10× microscope objective lens (Olympus PLN 10×; focal length, 18 mm) is put in front of the sample to emulate the eye lens. The central and peripheral subregions of the superior- and inferior-temporal retinal quadrants are imaged, where previous studies show that both Aβ and pTau pathologies preferentially appear in AD patients. In a subset of patients, respective brain section (BA9—frontal cortex) are imaged. Next, the imaged retinal region may be isolated, paraffin embedded, and sectioned. The cross sections may be further examined through immunohistochemistry (IHC). For ground-truth fluorescence imaging, the following primary monoclonal antibodies are utilized: anti-hAβ₄₂ (12F4), anti-hAβ₄₀ (11A5-B10), anti-total hAβ (6E10, 4G8), and anti-intracellular Aβ oligomers (scFvA13). Since studies of post-mortem AD brains indicate that certain pTau epitopes are directly related to early AD pathogenesis, neurotoxicity and cognitive decline, tissues using the following anti-pTau Abs are also examined: AT100 (pT212/pS214), AT8 (pS202/pT205), AT270 (pT175/pT181), pSer396 (AS-54977), and anti-PHF-1 (pS396/pS404). Secondary antibodies, conjugated with Cy-2, Cy-3, Cy-5 or DyLight™ 649, may be applied for fluorescent detection. Alternatively, signals may be detected with a highly sensitive immunoperoxidase methodology using DAB Substrate Chromogen System. In a subset of patients, a standard hematoxylin and eosin stain may be used to determine the cellular and nuclear location of Aβ and tau accumulation. To ensure that the component being detected in the tissue is not derived from the exogenous normal serum, the blocking step may be omitted for a subset of retinal and brain tissues. The primary antibody/antibodies may be omitted from adjacent sections as an IHC control. Fluorescence and bright-field images may be acquired under pre-defined conditions using a Zeiss Axio Imager Z1 fluorescence microscope.

We may register the retinal S-OCT cross-sectional images with histological fluorescence/DAB images by selecting one spectral channel image with a high contrast and register it with immunostained image using affine transformation, which is performed using rotation, translation, and scale to produce a non-reflective similarity transformation. If the affine registration is not sufficient by visual inspection, an optional control-point registration may be applied using control point pairs selected from the tissue, such as blood vessel edges. The control point registration may be implemented by a local weighted mean of inferred second degree polynomials from each neighboring control point pair to create a transformation mapping.

Quantification of Detection Accuracy of Retinal Aβ and pTau Deposits.

The output measure of the proposed snapshot S-OCT is spectrally resolved relative optical density images with a dimension of 400×400×13 (x, y, z). Because the spectrum-based classification may be performed at the image voxel level as described above, an image voxel can be referred to herein as a “sample.” A total of 32 AD/MCI subjects were imaged and stratified into two groups based on neuropathological and clinical status: training (AD patients, N=20) and validation (MCI patients, N=12). For subjects in the training group, at least six different fields of view per subject were captured, leading to a total of 12.5 million (400×400×13×6) samples. By combining all subjects in the training group, there is a total of 250 million samples. Provided that 1% of these samples can be labeled as either Aβ or pTau, respectively, through comparison to the immunofluorescence ground truth, the effective training samplings may be 2.5 million for respective substance. After the model is trained, it can be evaluated on the subjects in the validation group using the accuracy as the metric, which is defined as: accuracy=(TP+TN)/(TP+FN+TN+FP), where TP is true positive, TN is true negative, FP is false positive, and FN is false negative. The distributions of Aβ and pTau derived from snapshot S-OCT and fluorescence imaging voxel-wise can be compared, and the above metric can be calculated for each subject. With 12 subjects in the validation group and assuming each subject contributes at least one volumetric image containing the Aβ and pTau deposits, a power of 86% is obtained to demonstrate that the mean accuracy of snapshot S-OCT in detecting these deposits is as high as that for fluorescence imaging. This assumes that the mean accuracy for two modalities is precisely equal, that a difference of ten points or less is unimportant, and alpha is 0.05.

Additionally, a total of 12 non-AD healthy controls (N=12 retinas and N=6 paired brains) can be imaged, and the results obtained with snapshot S-OCT between the healthy controls and MCI subjects (N=12 retinas and N=6 paired brains) can be quantitatively compared. The superior- and inferior-temporal quadrants of retinal flatmounts can be imaged, in parallel to brain sections, by laterally scanning the sample, stitch the volumetric images acquired, and count the total number of image voxels that are classified as Aβ and pTau.

Assess Snapshot S-OCT in Transgenic APP/PS1 and hTau Mice In Vivo

Upon completion of ex vivo experiments, the system may be validated in vivo in the double-transgenic APP_{SWE}/PS1_{ΔE9} (APP/PS1)⁷⁷ and htau murine models of AD. Specifically, for in vivo imaging of retinal amyloidosis, double transgenic B6.Cg-Tg (APP_{SWE}/PS1_{ΔE9})85Dbo/J mice hemizygous for the transgenes and their age/sex-matched wild-type (WT) littermates can be used. For live imaging of retinal pTau, the transgenic model of tau may be used: B6.Cg-Mapt^{tm1(EGFP)Klt} Tg(MAPT)8cPdav/J hemizygous for the transgene and their age/sex-matched WT littermates. The distributions of retinal Aβ and pTau spectral deposits can be quantified, and the classification accuracy can be compared with the histological gold standard. This approach may be further validated by observing how an immune-based therapy alters retinal Aβ/pTau deposits in vivo. This may allow the technique to be validated in detecting in vivo retinal AD hallmarks and refine the imaging device, thereby laying the groundwork for the future human study.

### Imaging Procedure.

We may validate snapshot S-OCT in vivo using APP/PS1 and htau Tg mice described above, in which disease-associated Aβ and pTau have been both found in the retina. The mouse may be placed onto a 37° C. thermostatic heating pad, secured by a custom holder, and imaged under general anesthesia. The objective lens can be positioned vertically, and the eye of the mouse upwards can be faced upwards. For pupil dilation, one drop of ophthalmic solution (0.5% phenylephrine hydrochloride with 0.5% tropicamide) can be applied to the mouse eye. To suppress the strong optical front surface curvature of the mouse cornea and related spherical aberrations, a gel formulated from 2 mg/mL high molecular weight (4×10⁶ g/mole) carbomer in sterile 1× Dulbecco's PBS can be used to form the viscous optically transparent interface between the mouse cornea and a premium cover glass (Fisher Scientific), creating a Hruby-type lens for enhanced retinal imaging.

Validating Detection Accuracy of In Vivo Retinal Aβ and pTau Deposits.

To quantify the accuracy of the snapshot S-OCT system in detecting in vivo retinal Aβ and pTau deposits, AD-Tg mice from each mouse model (N=30) and WT control mice (N=30) can be imaged, equal numbers of male and female mice, at ascending ages of 3-10 months. After in vivo snapshot S-OCT measurement, the mouse may be euthanatized, and the posterior eye portion may be dissected out. Histopathology (IHC) and biochemical (ELISA/MSD) quantification of retinal pTau and Aβ may be compared to Snapshot S-OCT Imaging. The fluorescence-stained retinal cross sections and flatmounts may be imaged under a Carl Zeiss Axio Imager Z1 fluorescence microscope. Our preliminary data shows that the spectra of Aβ, pTau, and normal ocular tissue significantly differ in retinal cross section (FIGS. 3A, 3B, 4A, and 4B). Similar spectral difference may be detected from the in vivo retina as well. In each retinal cross section measured by snapshot S-OCT, the spectra measured at the ground-truth Aβ and pTau locations can be averaged and compared with that of the normal tissue through MANOVA test. Like snapshot S-OCT, in vivo AFI is a label-free technique that can provide amyloid-specific contrast. Therefore, the proposed technology can be compared against AFI. The mouse retina can be excited with a violet light LED (380 nm) and image the autofluorescence using the same system. The reference arm of the OCT system can be blocked, and the en-face retina can be directly imaged using the IMS. Next, all the spectral channel images acquired can be added to form a grayscale image and classify Aβ/pTau/normal tissue by thresholding the light intensity. For comparison, the classified snapshot S-October 3D image can be converted to a 2D image through maximum intensity projection along the depth axis.

Evaluation of the System in Monitoring the Retinal Aβ pTau Change During AD Immunotherapy

In some implementations, snapshot S-OCT can be evaluated as a longitudinal monitoring tool of disease progression and therapeutic response. For example, snapshot S-OCT can be used to repeatedly generate images of a retina during a time period when treatment is being performed. The total Aβ/pTau image voxels in the 3D snapshot S-OCT images can be determined to use as a metric to detect the reduction of retinal Aβ/pTau deposits after immunotherapy via snapshot OCT. The Aβ/pTau image voxels can be examined in the same field of view before and after immunotherapy.

In various embodiments, the method further comprises predicting cognitive decline in the subject. In various embodiments, the method further comprises monitoring the subject by repeating the method.

As described herein certain antibodies or compounds are labelled. As used herein, the term “label” refers to a composition capable of producing a detectable signal indicative of the presence of a target. Suitable labels, for example, for the anti-LRP-1 antibodies anti-PDGFR-β antibodies, and anti-Aβ compounds, include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods and devices described herein. For example, peptides can be labeled with a detectable tag which can be detected using an antibody specific to the label.

Exemplary fluorescent labeling reagents include, but are not limited to, Hydroxycoumarin, Succinimidyl ester, Aminocoumarin, Methoxycoumarin, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.

In some implementations, HSI images obtained using the example snapshot S-OCT system 509 shown in FIG. 5B can be further analyzed by a variety of machine learning models. These models can be used to classify the HSI images and generate abundance maps of constituent components, and further to transform the HSI images into images that resemble immunofluorescence-stained images and DAB-stained images. In some implementations, the HSI images can be input into a generative adversarial network (GAN) model to classify and transform the images. The GAN model is a competitive network that includes a generator and a discriminator. The discriminator is trained to classify real inputs and fake inputs that are generated by the generator. The GAN model is trained to transform unstained human retinal cross-sections into two types of standard histopathology images (immunofluorescence-stained images and DAB-stained images).

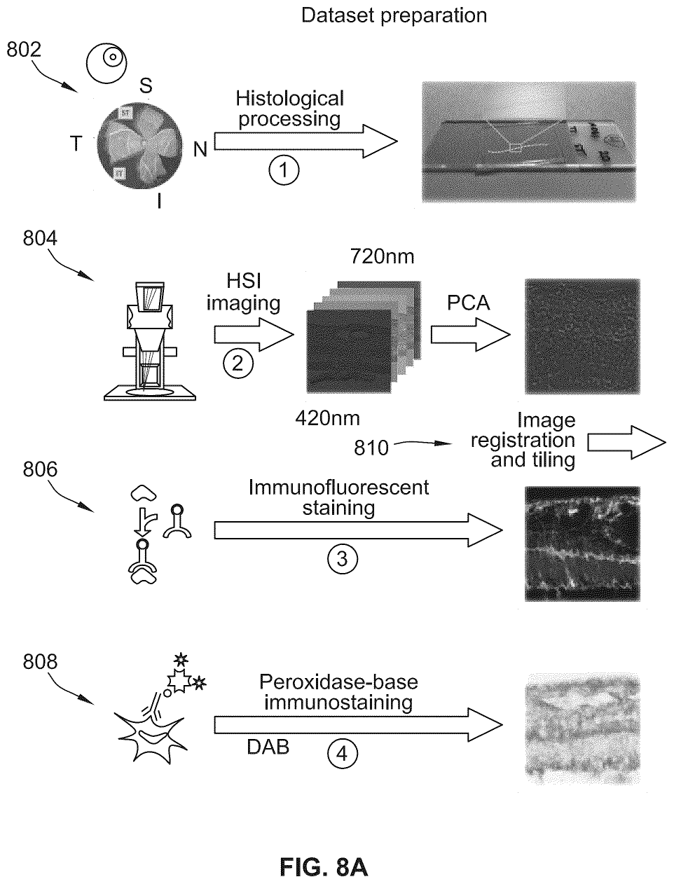

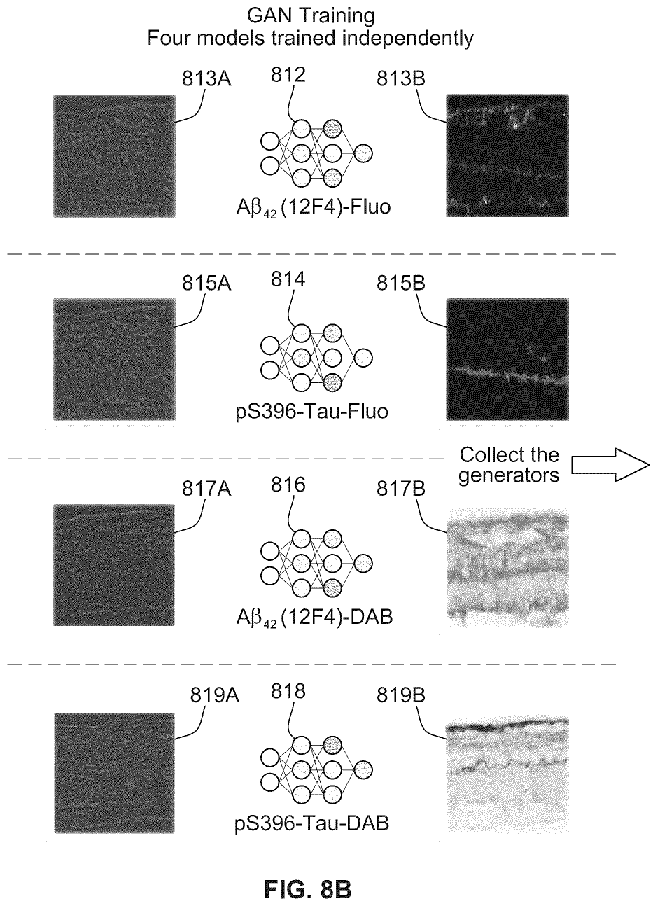

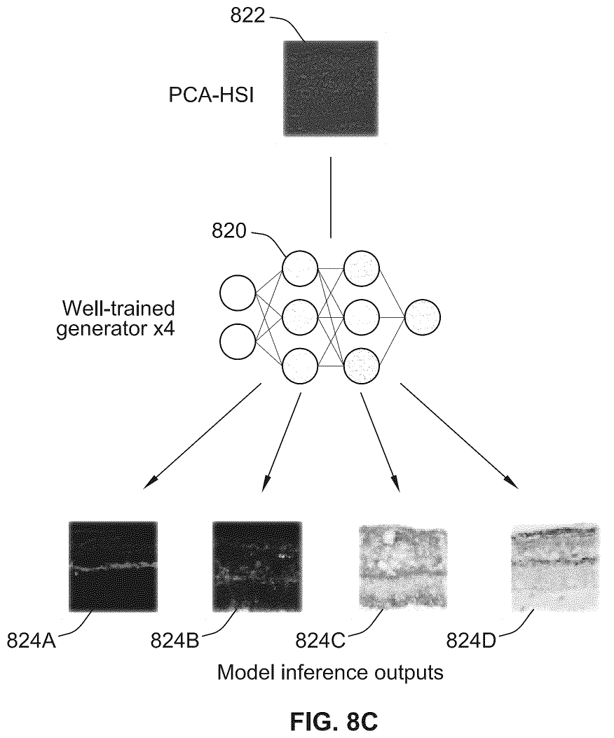

FIG. 8A shows the steps taken to prepare the inputs to train the GAN model. At step 802, retinal cross-sections are prepared by fixing the donor eye, isolating the retina to flatmounts, creating four retinal quadrants (S-superior; T-temporal; I-inferior; N=nasal), and sectioning the superior-temporal (ST) and inferior-temporal (IT) strips. At step 804, HSI images are obtained from the retinal cross-sections. In some implementations, HSI data is obtained in the form of a hypercube (x, y, λ). The hypercube can be converted to one or more three-channel HSI images by principal component analysis to represent the significant differences of the imaged pixel spectra, which can reduce the data load for training, while preserving most of the variability in the original hypercube. In some implementations, to obtain the HSI hypercube, the intensity at each spectral band is averaged over a selected area, and the intensity values are calibrated by pre-determined calibration coefficients. The overall intensity of the spectral range was then normalized. At step 806, immunofluroescent-stained samples of the retinal cross-sections are prepared and imaged. At step 808, DAB-stained (peroxidase-based immunostaining) samples of the retinal cross-sections are prepared and imaged. At step 810, the HSI images are registered with the immunofluorescent-stained images and the DAB-stained images. Patches of a suitable size (e.g., 256×256 pixels) are cropped from the HSI images and the stained images.

Referring now to FIG. 8B, the HSI image patches and the stained image patches are then formed into pairs, and four different GAN models are trained independently. GAN model 812 is trained using an HSI image patch 813A of Aβ deposits and the corresponding immunofluorescent-stained image patch 813B of Aβ deposits. GAN model 814 is trained using an HSI image patch 815A of pTau deposits and the corresponding immunofluorescent-stained image patch 815B of pTau deposits. GAN model 816 is trained using an HSI image patch 817A of Aβ deposits and the corresponding DAB-stained image patch 818B of Aβ deposits. GAN model 818 is trained using an HSI image patch 819A of pTau deposits and the corresponding DAB-stained image patch 819B of pTau deposits. In some cases, autofluoresence signals are found within the blood vessel lumen in the immunofluorescent-stained images. In some implementations, these lumen signals were removed by labeling them as negative, and enhancing the contrast of the true autofluoresence signals indicative of the Aβ deposits and the pTau deposits.

In some implementations, a structural similarity index (SSIM) component is incorporated into the generator loss function of the models as as −v×log[(1+SSIM(G(x), y))/2]. Mean absolute error (L₁) loss used to regularize the generator to transform the input image accurately and in high resolution. SSIM is used to balance the L₁ loss of learning correct features rather than the pixel accuracies. The loss function has the following form:

$$ℒ ( G ; D ) = - log D ( G ( x ) ) + λ \times {L}_{1} ( G ( x ) , y ) - v \times log [ \frac{1 + SSIM ( G ( x ) , y )}{2} ] ; \qquad Generator$$

$$ℒ ( D ; G ) = - log D ( y ) - log [ ( 1 - D ( G ( x ) ) ] ; \qquad Discriminator$$

where x is the PCA-compressed HSI image, y is the ground truth image, and G/D denotes the forward pass of the generator/discriminator network, λ and v are weights to control the loss of L₁ and SSIM terms. The network utilizes both spatial and spectral information to classify Aβ and pTau. In some implementations, the weights for the loss function components were set as 100 for L1 loss, and 100 for SSIM term. In some implementations, learning rates of 5×10⁻⁶ for the immunofluorescence models and 1×10⁻⁵ for the DAB models using the adaptive moment estimation (Adam) optimizer were used. In some implementations, the batch size was set to one under the instance normalization. In some implementations, the epoch number was in between 120 and 150, with 50 epochs for decayed learning rate. In some implementations, training time was approximately 47 h for immunofluorescence models and 82 h for DAB models.

The result of the training is the trained model 820 shown in FIG. 8C. With the trained model, an HSI image 822 (which may be obtained by converting an HSI hypercube to a three-channel HSI image via principal component analysis) can be input into the model 820 (which can be a single model or multiple separate models0, which outputs an immunofluorescent-stained image 824A of the pTau deposits, an immunofluorescent-stained image 824B of the Aβ deposits, a DAB-stained image 824C of the pTau deposits, and a DAB-stained image 824D of the Aβ deposits. The trained model 820 is referred to as a single model, but can generally be four separate trained models. The stained images 824A-824D can be considered as virtualized stained images, as they are not images taken directly from a retinal sample that has been extracted and stained. Thus, the trained model 820 (which may be multiple separate trained models) can produce stained images of a subject's retina without having to extract and stain a physical sample of the subject's retina. These virtualized images 824A-824D can be used to determine biomarkers that are indicative of AD (e.g., the presence and/or amount of Aβ and/or pTau deposits in a subject's retina).

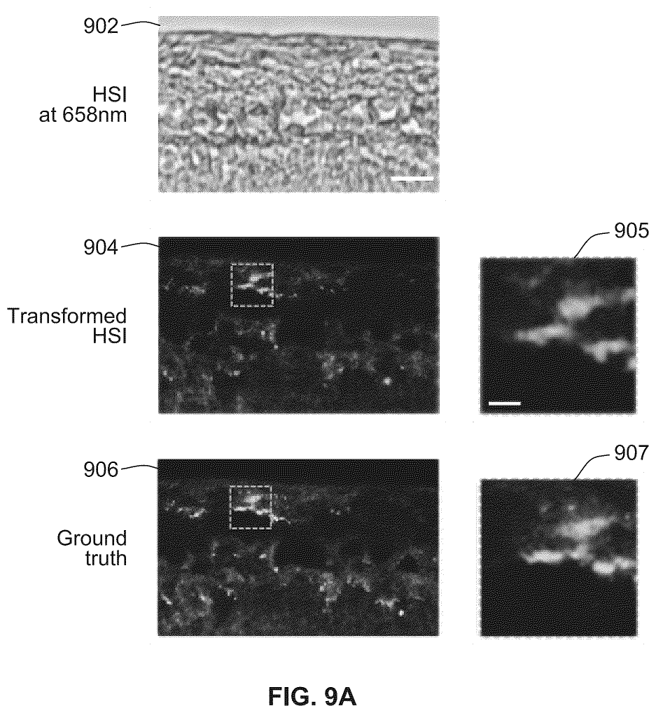

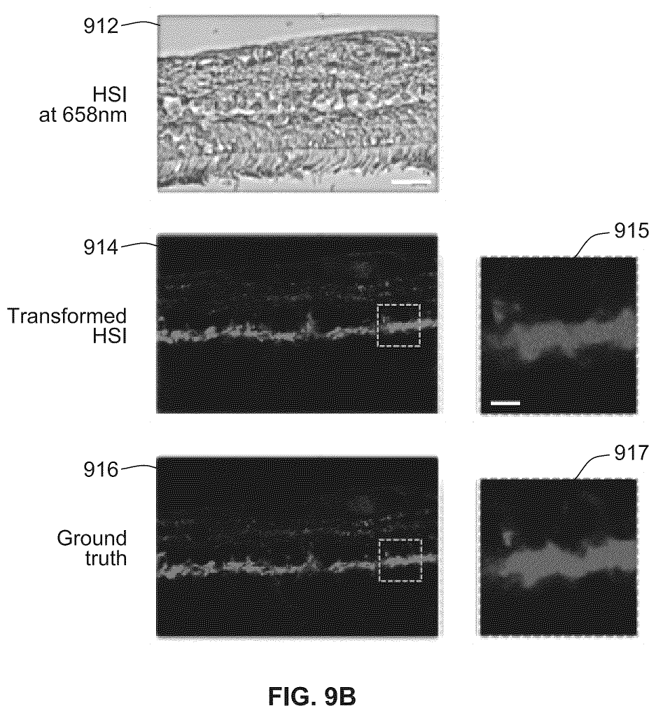

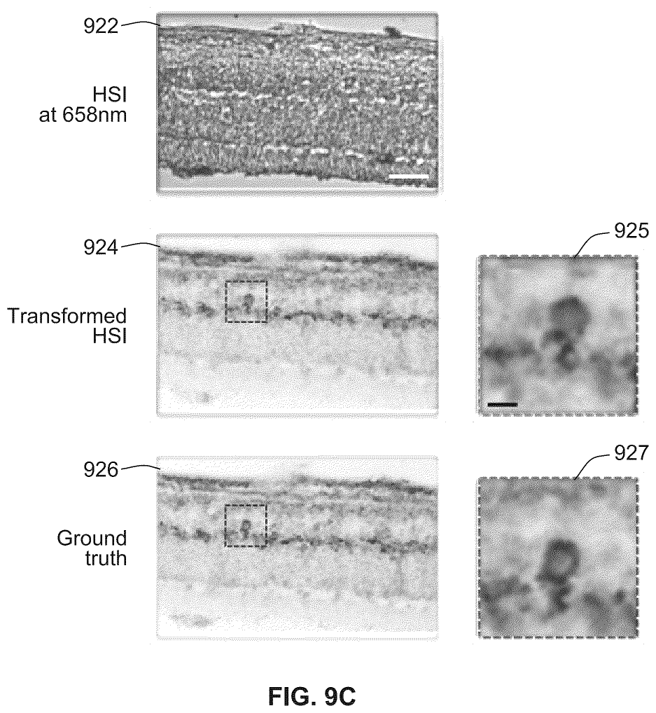

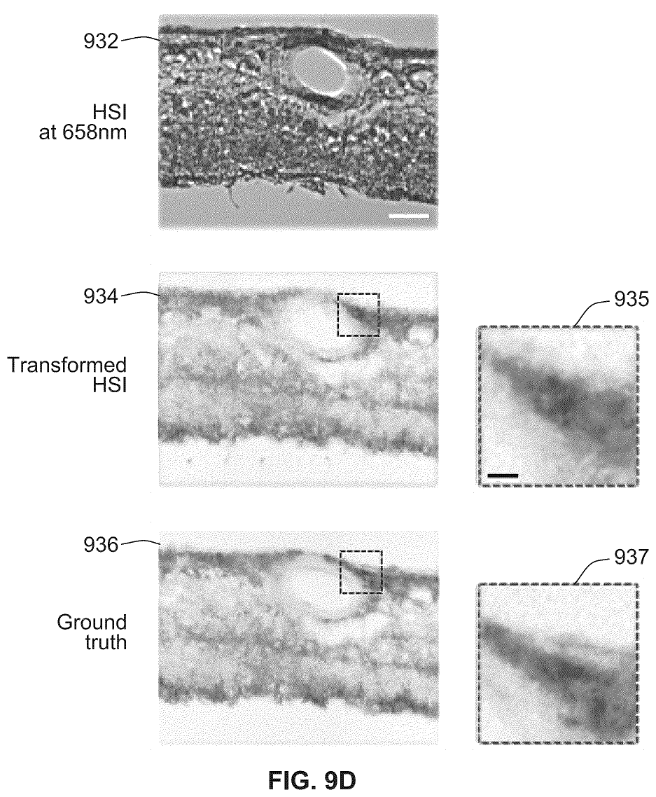

FIGS. 9A-9D show the results of the trained model 820. FIG. 9A shows an HSI image 902 of an Aβ deposit that is input into the trained model 820, a transformed HSI image 904 output from the trained model 820 that is made to look like an immunofluorescent-stained image of the Aβ deposit, and a ground truth immunofluorescent-stained image 906 showing the Aβ deposit that is used for comparison. The ground truth image 906 was obtained by performing the immunofluorescent-staining process on the sample retinal cross-section. As can be seen, the ground truth image 906 compares favorably to the transformed HSI image 904. The transformed HSI image 904 includes zoomed-in portion 905, and the ground truth image 906 include a zoomed-in portion 907, both showing the same specific feature. Again, the zoomed-in portion 905 of the transformed HSI image 904 compares favorably to the zoomed-in portion 907 of the ground truth image 906.

FIGS. 9B-9D each show a similar series of images as FIG. 9A. FIG. 9B shows an HSI image 912 of a pTau deposit that is input into the trained model 820, a transformed HSI image 914 output from the trained model 820 that is made to look like an immunofluorescent-stained image of the pTau deposit, a zoomed-in portion 915 of the transformed HSI image 914, a ground truth immunofluorescent-stained image 916 showing the pTau deposit, and a zoomed-in portion 917 of the ground truth image 916.

FIG. 9C shows an HSI image 922 of an Aβ deposit that is input into the trained model 820, a transformed HSI image 924 output from the trained model 820 that is made to look like a DAB-stained image of the Aβ deposit, a zoomed-in portion 925 of the transformed HSI image 924, a ground truth DAB-stained image 926 showing the Aβ deposit, and a zoomed-in portion 927 of the ground truth image 926.

FIG. 9D shows an HSI image 932 of a pTau deposit that is input into the trained model 820, a transformed HSI image 934 output from the trained model 920 that is made to look like a DAB-stained image of the pTau deposit, a zoomed-in portion 935 of the transformed HSI image 934, a ground truth DAB-stained image 936 showing the pTau deposit, and a zoomed-in portion 937 of the ground truth image 936. As can be seen, the transformed HSI images 914, 924, and 934 (and their zoomed-in portions 915, 925, and 935) compare favorable to the corresponding ground truth images 916, 926, and 936 (and their zoomed-in portions 917, 927, and 937).

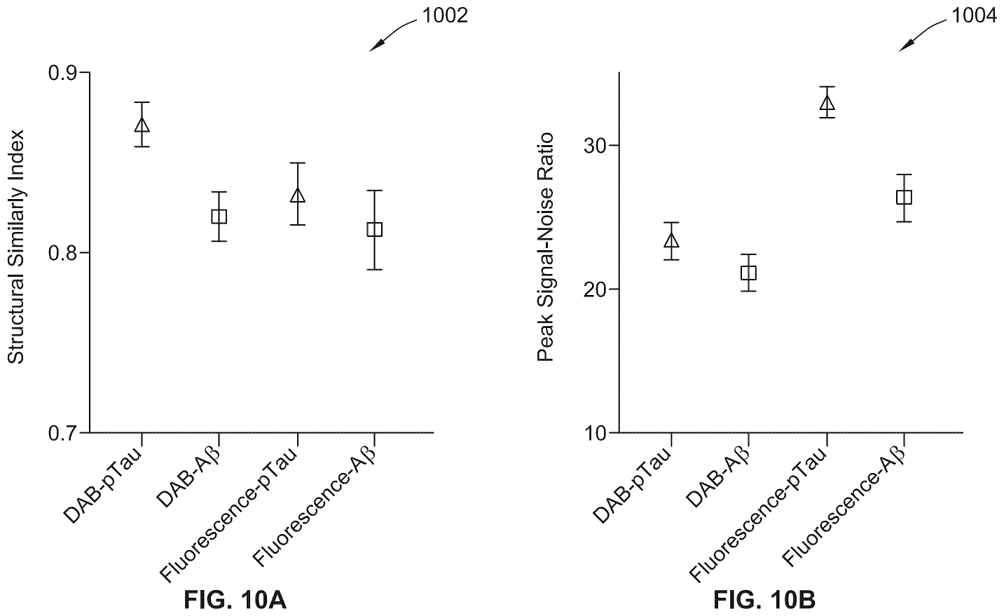

FIG. 10A shows a structural similarity index (SSIM) plot 1002 that assesses the similarity between the transformed HSI images 904, 914, 924, 934 and the corresponding ground truth images 906, 916, 926, and 936. SSIM is a perception-based image quality metric which evaluates structural similarities between synthesized images in deep-learning methods. An SSIM of one is a perfect match, while a zero indicates a severe dissimilarity. The SSIM plot 1002 shows the SSIM values for the four components of the trained model 820: DAB-pTau, DAB-Aβ, immunofluorescent-pTau, and immunofluorescent-Aβ. As shown, each of the four components has a favorable SSIM value. Thus, the SSIM plot 1002 shows that the trained model 820 can successfully recover the staining color scheme, and can discriminate retinal Aβ and pTau deposits.

In some implementations, the SSIM metric between a transformed HSI image/and a ground truth image j is calculated using the following equation:

$$SSIM ( i , j ) = \frac{( 2 {μ}_{i} {μ}_{j} + {c}_{1} ) ( 2 {σ}_{i j} + {c}_{2} )}{( {μ}_{i}^{2} + {μ}_{j}^{2} + {c}_{1} ) ( {σ}_{i}^{2} + {σ}_{j}^{2} + {c}_{2} )}$$

where μᵢ and μⱼ are the averages of i and j; σᵢ and σⱼ are the standard deviations of i and j; σᵢⱼ is the covariance of i and j; and c₁ and c₂ are regularization constants to avoid instability when the other variables are close to zero.

FIG. 10B shows a peak signal-to-noise ratio (PSNR) plot 1004 that evaluates the image quality of the transformed HSI images 904, 914, 924, and 934. As can be seen, each of the four components of the trained model 820 have a PSNR value that is greater than 20 dB, which indicates high image quality.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “control system” on data stored on one or more computer-readable storage devices or received from other sources.

The term “control system” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it may be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It may be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It may be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A retinal imaging system for obtaining one or more retinal images of a retina of a subject, the system comprising:
an in vivo retinal imaging modality; and
an image mapping spectrometry (IMS) camera integrated with the in vivo retinal imaging modality;
wherein the retinal imaging system is configured to image inner and outer retinal cell layers; and
wherein the retinal imaging system is configured to acquire the one or more retinal images in a snapshot format.

2. The retinal imaging system of claim 1, wherein the in vivo retinal imaging modality is a spectroscopic optical coherence tomography (S-OCT) system.

3. The retinal imaging system of claim 1, wherein the IMS camera includes an image mapper comprising a number of mirror facets, each of the number of mirror facets having a two-dimensional tilt angle.

4. The retinal imaging system of claim 3, wherein the number of mirror facets is based on a desired depth range for acquiring inner retinal layers.

5. The retinal imaging system of claim 4, wherein the number of mirror facets is in a range between 100 and 600 mirror facets.

6. The retinal imaging system of claim 3, wherein the IMS camera includes a high-resolution grating, an array of lenslets, and a detector array configured to measure full field spectral interferograms.

7. The retinal imaging system of claim 1, wherein the one or more retinal images include a plurality of spectrally resolved volumetric images of the retina of the subject at one or more wavelengths in a wavelength range between 500 nm and 650 nm.

8. The retinal imaging system of claim 1, wherein the in vivo retinal imaging modality comprises a water vial for dispersion balancing the subject's eye for in vivo imaging.

9. The retinal imaging system of claim 3, wherein the IMS camera is a high-spectral resolution IMS camera, or wherein the IMS camera comprises a plurality of low-spectral resolution IMS cameras that each measure a separate spectral range.

10-11. (canceled)

12. The retinal imaging system of claim 1, wherein the retinal imaging system is configured to spectrally resolve spectral signatures of amyloid β deposits in the retina of the subject, hyperphosphorylated pTau deposits in the retina of the subject, or both.

13. The retinal imaging system of claim 1, further comprising a light source emitting light in the visible spectrum.

14. A method for detecting biomarkers of Alzheimer's disease (AD) in vivo in a subject, the method comprising:
obtaining, via a full-field Fourier domain optical coherence tomography integrated with an image mapping spectrometry (IMS), image data reproducible as one or more retinal images of a retina of the subject;
detecting biomarkers of AD at spectral resolution wavelengths in a range between 500 nm and 650 nm from the image data.

15. The method of claim 14, further comprising generating a plurality of volumetric spectrally resolved retinal images of the subject from the image data.

16. The method of claim 15, wherein the plurality of volumetric spectrally resolved retinal images are obtained from a single snapshot acquisition.

17. The method of claim 14, wherein the biomarkers of AD include a presence of an amyloid beta (Aβ) deposit in the retina of the subject, a presence of a hyperphosphorylated pTau deposit in the retina of the subject, or both.

18. The method of claim 14, wherein the IMS camera is a high-spectral resolution IMS camera having a number of mirror facets corresponding to a desired depth range for imaging the retina of the subject, the desired depth range being between 20 micrometers and 200 micrometers.

19. (canceled)

20. The method of claim 14, wherein detecting the biomarkers of AD includes inputting at least a portion of the image data into one or more trained models, the one or more trained models being configured to output one or more virtualized stained images of the retina of the subject.

21. The method of claim 20, wherein the one or more trained model includes a single trained model configured to output a plurality of types of virtualized stained images of the retina of the subject, or a plurality of trained models that are each configured to output at least one type of virtualized stained image of the retina of the subject.

22. (canceled)

23. The method of claim 20, wherein the one or more virtualized stained images of the retina of the subject includes one or more virtualized immunofluorescent-stained images of the retina of the subject, one or more virtualized DAB-stained images of the retina of the subject, or both.

24-25. (canceled)

26. The method of claim 23, wherein the biomarkers of AD include a presence of the Aβ deposit in the retina of the subject, a presence of the hyperphosphorylated pTau deposit in the retina of the subject, or both.

27-31. (canceled)
